(19) United States
(12) Patent Application Publication
(10) Pub. No.: US 2003/0130573 A1
(43) Pub. Date: Jul. 10, 2003

(54) Method and device for optimization of preloaded brachytherapy needles

(76) Inventors: Yan Yu, Rochester, NY (US); Robert C. Jackson, Williamsville, NY (US)

Correspondence Address: BLANK ROME COMISKY & MCCAULEY, LLP, 900 17TH STREET, N.W., SUITE 1000, WASHINGTON, DC, 20006, US

(21) Appl. No.: 10/205,607

(22) Filed: Jul. 26, 2002

Related U.S. Application Data
(60) Provisional application No. 60/308,588, filed on Jul. 31, 2001.

(51) Int. Cl. A61B 5/05

(52) U.S. Cl. 600/407

(57) ABSTRACT

In prostate brachytherapy or the like, a preplan is formed for the prostate in its condition at the time, and needles pre-loaded with radioactive seeds are ordered. In the operating room, it is determined whether the prostate has changed in size, shape or position. If so, the preplan is deformed to conform to the prostate in its new condition. The needles are inserted into the prostate through a template, which can have a hole spacing in each dimension that is smaller than that that of conventional templates or can have holes arranged in a non-rectilinear pattern. Alternatively, a virtual template, having a single movable needle passage, can be used. The therapeutic agents can be provided in a biodegradable carrier for timed release.

### REFERENCE TO RELATED APPLICATION

The present application claims the benefit of U.S. Provisional Application No. 60/308,588, filed Jul. 31, 2001, whose disclosure is hereby incorporated by reference in its entirety into the present disclosure.

### FIELD OF THE INVENTION

The present invention is directed to optimization of placement of a set of radioactive sources for brachytherapy or the like and more particularly to such an optimization which can be performed in the operating room at the time of implantation.

### DESCRIPTION OF RELATED ART

Prostate brachytherapy is a form of therapy for prostate cancer in which radioactive sources (seeds, wires etc.) are loaded into needles which are then inserted into the patient's prostate in order to irradiate the cancer. Currently, many practitioners of prostate brachytherapy utilize a pre-load procedure which is not compatible with intra-operative. planning. Those practitioners generally create a “preplan” based on trans-rectal ultrasound images of the patient's prostate anatomy. The “preplan” includes the type of radioactive sources to be used (e.g. I -125 or Pd-103), the activity of the sources, specifications regarding how the sources should be loaded into a set of brachytherapy needles (i.e. the number of sources in each needle and their spacing) and the template coordinates for the placement of each needle.

In the pre-load procedure, each needle is loaded with the specified number of appropriately spaced radioactive sources prior to surgical implantation. The advantage of the pre-load procedure is that no time is expended loading sources into needles in the operating room. Therefore, pre-load procedures can be completed in less time and with less expense than intraoperative procedures which require loading of needles in the operating room. Use of factory-preloaded needles decreases handling of radio-isotopes by medical personnel and reduces radiation exposure levels.

A disadvantage of the pre-load procedure, as currently practiced, is that the “pre-plan” can be extremely difficult to reproduce and implement in the operating room. For proper image registration, the patient's surgical position must be identical to his pre-planning position. Proper positioning is difficult to achieve and is often complicated by differences in the physical setups in the operating room and ultrasound suite where TRUS is performed during the pre-planning visit. Hormone therapy-induced changes in the size of the prostate during the interval between pre-planning and implementation is another potential source of difficulty, as are changes in the shape of the prostate resulting from muscle relaxation under anesthesia. (No anesthesia is used during pre-planning TRUS.) These differences in the position, size and shape of the prostate can be substantial and are a source of concern for practitioners.

Another disadvantage of the pre-load procedure is that it is inflexible and cannot easily adjust for these differences. Using pre-loaded needles “as is” is incompatible with intra-operative planning and can seriously compromise the procedure. Yet, a great deal of practitioner experience is required to make high-quality, ad hoc plan changes, constrained to the specifications of the pre-loaded needles, in the pressurized atmosphere of the operating room.

One of the present inventors (Yan Yu) has previously developed a brachytherapy planning system known as PIPER-I and disclosed in U.S. Pat. No. 6,200,255, whose disclosure is hereby incorporated by reference in its entirety into the present disclosure. Briefly described, PIPER-I implements a synergistic formulation of a genetic algorithm, multi-objective decision theory and a statistical sensitivity analysis. In a preferred embodiment of this previous invention, a batch of radioactive sources is periodically ordered for a stream of patients, and eligible patients are then scheduled for implantation without any delays due to preoperative planning and source ordering. Each patient is then set up for surgery, and the TRUS (transrectal ultrasound) volume study and treatment planning steps are performed intraoperatively, i.e. at the time of the surgical procedure. With intraoperative planning, changes in the size, shape and position of the prostate are not a problem, because plans are created minutes before source implantation and are customized to the patient's actual surgical position.

In its planning aspects, the PIPER-I system reads the anatomy data previously generated and determines the maximum extent of the prostate size, as well as the degree of pubic arch interference. The preference profile of the clinician-user is then read from a profile file and such data is used to influence the baseline priorities of optimizing different objectives, such as the dosing of the prostate, keeping the number of needles to a minimum, etc. The dosimetry values are then looked up for the chosen seed type. Then, the two-dimensional genetic algorithm for the prostate is encoded, thus encoding the location of all potential needle placement positions, using a two-dimensional binary pattern. A population pool with a random population is then initiated, and the dosimetry for each member of the population is evaluated. Members of the population are then ranked using multi-objective metrics, and a dynamic n-tournament analysis is performed on the ranked members. A two-dimensional crossover and a mutation are then performed in turn. This entire process is repeated for a user-specified number of iterations, and a set of optimized planning solutions is presented to the user.

As commercially configured, the PIPER-I Brachytherapy Planning System is capable of generating an optimized brachytherapy plan any time prior to surgical implantation. However, plans generated by the PIPER-I system are created de novo, i.e. from individual radioactive sources and needles, not from a set of pre-loaded needles. As a consequence, while the PIPER-I system avoids the above-noted disadvantage of the pre-load procedure, it also avoids the above-noted advantage of the pre-load procedure.

### SUMMARY OF THE INVENTION

It will be readily apparent from the above that a need exists in the art to combine the advantages of PIPER-I and the pre-load procedure. It is therefore an object of the invention to allow practitioners to create, in the operating room at the time of implantation, a plan for the optimal placement of a set of brachytherapy needles which have previously been loaded with radioactive sources.

To achieve the above and other objects, the present invention is directed to a method and device for optimization of preloaded needles for brachytherapy or the like in which the genetic algorithm-based planning engine of the PIPER-I system is modified to optimize the placement of a set of brachytherapy needles that have previously been loaded with radioactive sources. Optimization can be accomplished by modifying an existing plan or by creating a new plan which utilizes the pre-loaded set of needles.

A coding system is used to transmit the specifications of the pre-loaded needle set to the planning system and ensure that only the correct set of pre-loaded needles is used in optimization. Through the coding system, use of the pre-load optimization feature can be limited to radioactive sources and/or pre-loaded needle sets provided by a specific manufacturer.

In order to provide flexibility and increase the degree of optimization achievable with a pre-loaded set of needles, the optimization engine can be designed to allow the user to have the option of specifying a number of extra needles and radioactive sources (the over supply, OS) in addition to the pre-loaded needle set, that may be used in generating the intraoperative plan. Those extra needles can be either pre-loaded with radioactive sources or custom loaded in the operating room at the time of the brachytherapy procedure.

Another way to increase the degree of optimization achievable by the present invention is through the use of enhanced template technology which allows needles to be inserted at various angles and with no constraints with regard to needle insertion position. Such templates afford the greatest degree of freedom for optimization.

To implement the pre-load technology, in at least one preferred embodiment, PIPER's genetic algorithm-based planning engine was substantially modified so the system can be used to optimize the placement of a set of brachytherapy needles that have previously been loaded with radioactive seeds. A coding system will be used to limit use of the pre-load technology module to PIPER-compatible seeds and preloaded needle sets. Through this coding system, use of the pre-load optimization feature can be limited to seeds and pre-loaded needle sets provided by a specific manufacturer.

In order to provide flexibility and increase the degree of optimization achievable with a pre-loaded set of needles, PIPER's pre-load optimization engine has been designed to allow the user to have the option of (1) not depositing all seeds in a particular needle and/or (2) specifying a number of extra needles and/or seeds (in addition to the pre-loaded needle set) that may be used in generating the intraoperative plan. These extra needles can be either pre-loaded with seeds or custom loaded in the operating room. The PIPER System can also be designed to create optimized plans using one of a number of generic sets of pre-loaded needles, each covering a range of prostate sizes, thereby potentially eliminating the need for creation of a preplan.

Another way to increase the degree of optimization achievable is through the use of enhanced template technology. The templates currently in use in prostate brachytherapy procedures are essentially flat plates with a 13×13 array of needle insertion holes perpendicular to the plane of the template and spaced 0.5 cm on center. Since needles can only be placed where there is a hole in the template, the ability of PIPER (or any other computerized planning system) to produce an optimized plan for needle and seed placement is constrained by the number, position and perpendicular angle of holes in the template.

The degree of potential optimization of any plan, whether constrained by the use of a specific set of pre-loaded needles or not, can be improved by decreasing the distance between potential needle placement positions and increasing their number. For instance a 26×26-hole template can be designed with 0.25 cm between each needle insertion hole. This 26×26-hole template would cover the same surface area as the conventional 13×13-hole template but would have four times as many potential needle insertion positions. In the most general form, templates can be designed that allow needles to be inserted at various angles and with no constraints with regard to needle insertion position. Such templates afford the greatest degree of freedom for optimization.

The benefits of this new PIPER technology include the following:

The many practitioners who use the pre-load procedure will be able to conduct intra-operative planning and optimization without changing their practice style and without increasing OR time; consequently, more patients will benefit from intra-operative planning and optimization.

Use of pre-loaded needles with intra-operative optimization is faster, more convenient and more efficient than any form of intra-operative loading, including all forms of automated loading devices.

Practitioners conducting intra-operative brachytherapy will probably switch to the pre-load methodology since it provides the benefits of intra-operative planning and optimization without the costs associated with intra-operative needle loading.

Unlike automated seed loading devices there is no capital equipment expense associated with the use of pre-loaded needles.

The pre-load feature of the PIPER System can add value to a particular seed manufacturer's radioactive seed products and stimulate sales.

Through the coding system, use of the pre-load optimization feature can be limited to seeds or pre-loaded needle sets provided by a specific manufacturer, thereby differentiating that manufacturer's seed products from those of competitors.

Use of factory pre-loaded needles will decrease handling of radioisotopes by medical personnel and reduce radiation exposure levels.

Use of factory pre-loaded needles is more convenient and efficient than hospital pre-loads.

Various preferred embodiments of the present invention have the following features:

1. A computerized planning system that can optimize placement of a set of brachytherapy needles that have previously been loaded with radioactive sources or other therapeutic agents of a multiplicity of strengths/concentrations and therapy release schedules.

2. A modification of the computerized planning system which allows the user to specify (and/or the computer system to recommend) an additional number of sources/needles to be used as an over supply inventory to supplement the set of pre-loaded needles in generating optimized needle placement plans that more closely achieve the clinician's planning objectives.

3. A modification of the computerized planning system which allows the creation of optimized plans using one of a number of generic sets of pre-loaded needles, each covering a range of prostate sizes, thereby eliminating the need for creation of a preplan.

4. The use of a code to communicate to the computerized planning system that a specific seed type or set of pre-loaded brachytherapy needles are suitable for use in intraoperative planning.

5. A brachytherapy template which contains a larger number of more closely spaced needle placement positions (holes) than the conventional 13×13-hole template with 0.5 cm on center needle insertion holes.

6. A virtual template that allows needles to be inserted at various angles and with no constraints with regard to needle insertion position.

7. A force-sensing actuator working in conjunction with the virtual template to place or guide the placement of each needle using a variety of forces and needle rotation patterns.

The computations required for the present invention can be implemented on any suitable device, such as a commercially available microcomputer with a Pentium II or higher microprocessor.

### BRIEF DESCRIPTION OF THE DRAWINGS

Preferred embodiments of the present invention will be set forth in detail with reference to the drawings, in which:

FIG. 1 is a flow chart showing a needle plan optimization process according to a preferred embodiment of the present invention;

FIGS. 2A-2D are drawings showing treatment plans according to various stages of the process of FIG. 1 superimposed on an outline of the patient's prostate;

FIG. 3 is a flow chart showing an implementation of the modification step in the needle plan optimization process of FIG. 1;

FIGS. 4A-4D are drawings showing a variation in the needle placement plan through crossover;

FIGS. 5A and 5B are drawings showing a variation in the needle placement plan through mutation;

FIG. 6 is a drawing showing a variation in the needle placement plan through migration;

FIG. 7A is a drawing showing a template according to the prior art;

FIG. 7B is a drawing showing a template in which the distance between insertion locations is decreased and the number of insertion locations is increased relative to the standard template shown in FIG. 7A;

FIGS. 7C-7E are drawings showing a template with a multiplicity of non-uniformly spaced, fixed needle insertion locations some of which allow needles to be inserted at various angles according to a preferred embodiment of the present invention;

FIG. 7F is a drawing showing a virtual template according to another preferred embodiment of the invention;

FIG. 8 is a block diagram of a computer system for carrying out any of the preferred embodiments of the invention;

FIG. 9 is a graph showing the minimum peripheral dose data for fourteen patients;

FIG. 10 is a graph showing the D95 data for the same fourteen patients;

FIG. 11 is a screen shot showing an isodose distribution for a plan that has not been optimized in the OR;

FIG. 12 is a screen shot showing the DVH data corresponding to FIG. 11;

FIG. 13 is a screen shot corresponding to FIG. 11, except after optimization;

FIG. 14 is a screen shot corresponding to FIG. 12, except after optimization; and

FIG. 15 shows a partial cross-sectional view of a carrier for therapeutic agents usable with one embodiment of the present invention.

### DETAILED DESCRIPTION OF THE PREFERRED EMBODIMENTS

Preferred embodiments of the present invention will now be set forth in detail with reference to the drawings. It should be understood throughout that any technical details may be incorporated from the present inventor's above-cited previous patent as needed.

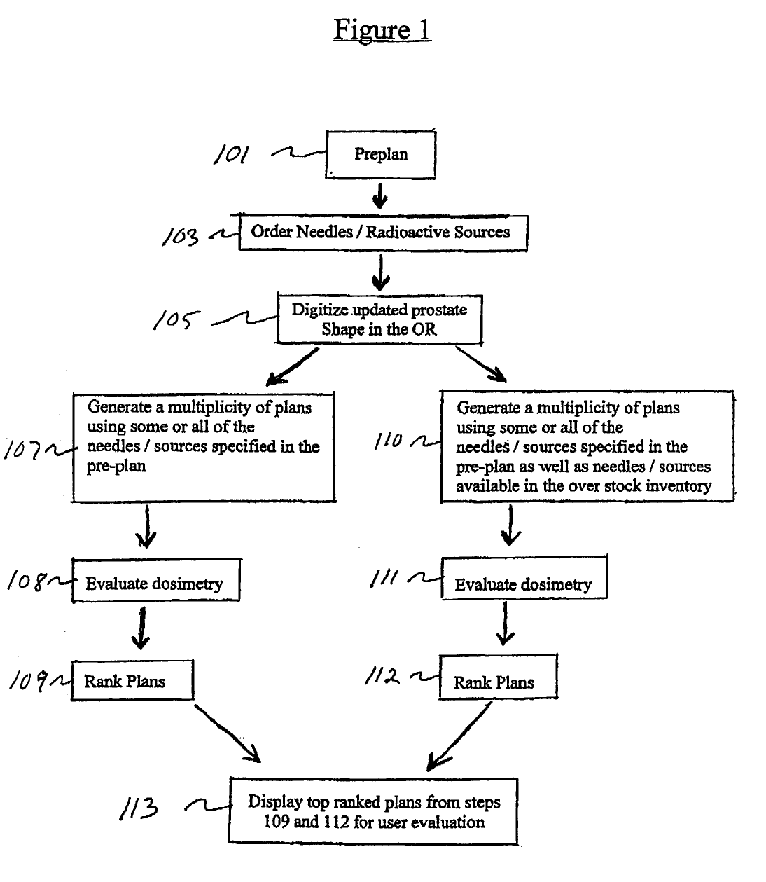

FIG. 1 is a flow chart showing an overview of a needle plan optimization process according to a first preferred embodiment of the present invention. A preplan is performed in step 101 for a prostate brachytherapy implant by one of several techniques (manual planning, geometric planning, any optimization or inverse planning method, or nomogram and rules of thumb). A set of needles that have been loaded with radioactive sources are then ordered in step 103. The radioactive source/needle pattern, i.e. the type of radioactive sources used to load the needles (e.g. I-125 or Pd-103), the activity of the sources, specifications regarding how the sources should be loaded into each needle (i.e. the number of sources in each needle and their spacing) and the template coordinates for the placement of each loaded needle are specified by the preplan. At the time of implantation, the prostate shape is updated and digitized in the operating room (OR) in step 105 utilizing a suitable imaging technique such as ultrasound. Another plan (“intraop plan”) may be required because of differences in the position, size and shape of the prostate which can occur in the interval between creation of the preplan in step 101 and the surgical implantation of radioactive sources.

After step 105, the flow chart of FIG. 1 branches out into two sub-processes, including steps 107-109 and 110-112 respectively. Either or both of the sub-processes can be carried out.

A multiplicity of potentially suitable intraop plans are created in steps 107 and 110. Plans created in step 107 use some or all of the set of preloaded needles specified in the preplan. Plans created in step 110 use all or some of the set of preloaded needles but may also use the OS inventory. The multiplicity of potentially suitable plans created in steps 107 and 110 are generated by modifying the preplan through a process of mathematical optimization to match the updated prostate shape and size digitized in step 105. In steps 108 and 111, the dosimetry of the potentially suitable plans generated in steps 107 and 110, respectively, is evaluated. In step 109, the plans generated in step 107 are ranked using multi-objective metrics. In step 112 the plans generated in step 110 are ranked using multi-objective metrics. In step 113, a selection of the highest ranked plans from step 109 or 112, or from both of steps 109 and 112 if both sub-processes are carried out, is presented to the user for evaluation.

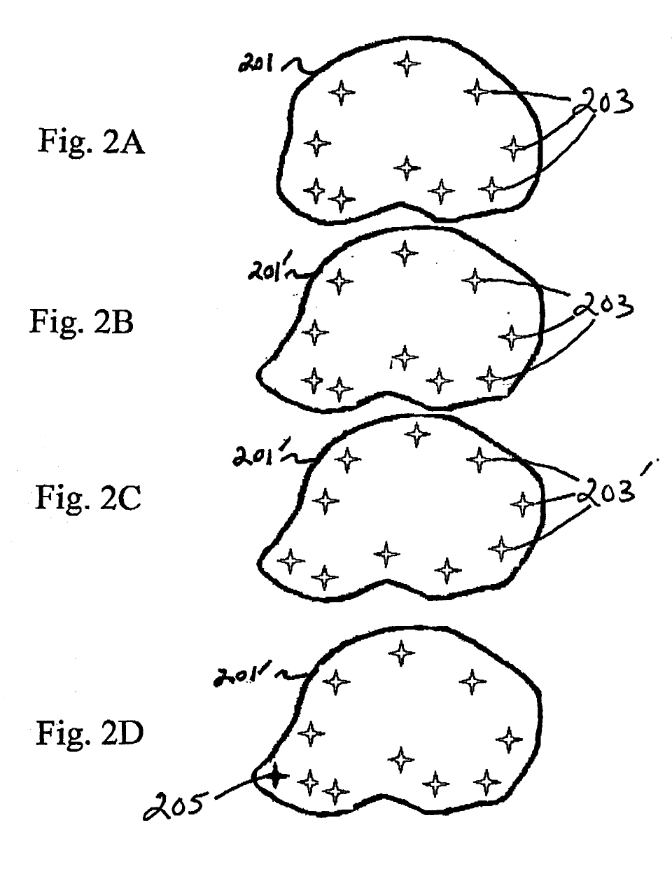

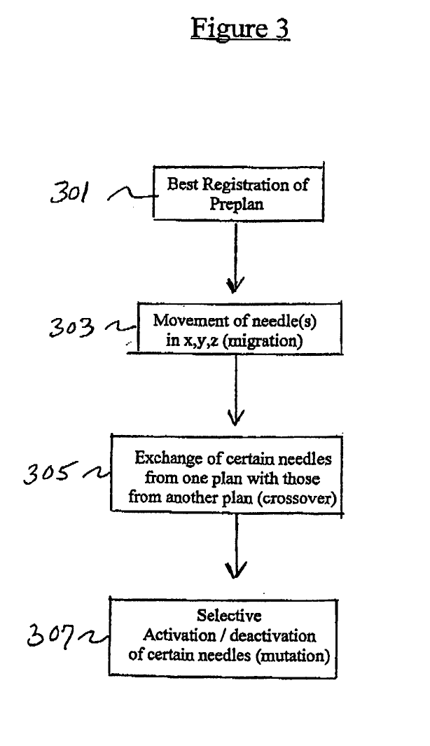

The process of FIG. 1 will be illustrated with the examples shown in FIGS. 2A-2D. FIG. 2A shows a preplan produced by step 101 of FIG. 1, in which an outline 201 of the patient's prostate is shown with needle locations 203. FIG. 2B shows the updated outline 201′ of the patient's prostate, as determined in step 105 of FIG. 1, with the needle locations 203 from the preplan superimposed thereon. FIG. 2C shows one of the multiplicity of potential intraop plans produced in step 107 of FIG. 1, in which modification of the preplan moves the needle locations 203 to new locations 203′. FIG. 2D shows one of the multiplicity of potential intraop plans produced in step 110 of FIG. 1 in which a needle from the OS inventory, 205, is used in addition to some/all of the needles from the prepalan.

FIG. 3 is a flow chart showing the manner in which the plan modifications of FIG. 1, steps 107 and 110 are carried out. The modification operation uses the following steps:

Step 301. Best registration of preplan onto the intraop prostate: requires centering and containment (described below).

Step 303. Movement of at least one needle by one or more increments in the x, y, and z directions (migration): requires containment.

Step 305. Exchange of certain needles from one plan with those from another plan (crossover): requires containment.

Step 307. Selective deletion/addition of certain needles (mutation).Steps 303, 305 and 307 should be concurrently operating (e.g., by using a genetic algorithm or simulated annealing-genetic algorithm hybrid optimization). The use of a genetic algorithm to produce a placement plan and in particular to optimize dosimetry is described in the above-cited patent. Details of an implementation of a genetic algorithm (GA) to effect steps 303, 305 and 307 include crossover, mutation and migration, which will be described below.

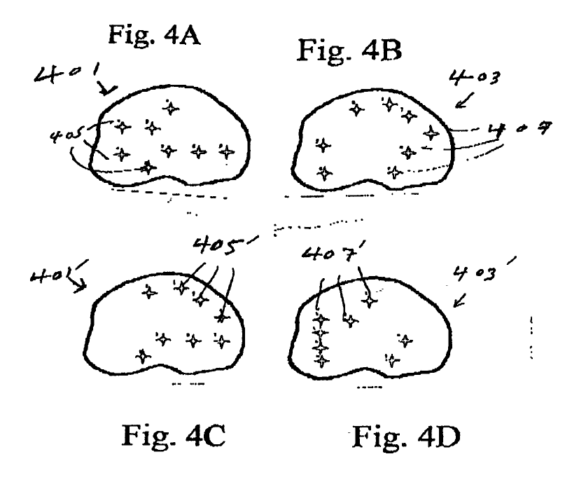

Crossover will be explained with reference to FIGS. 4A-4D. Needles in the available inventory (preplan pattern plus over supply) are labeled uniquely, e.g., 1, 2, 3, . . . Crossover operates by exchanging the randomly selected needle pairs (approx. ½ of the total needles) between two plans.

FIGS. 4A and 4B show two randomly selected intraop plan candidates, 401 and 403, from one generation, with needle positions 405 and 407, respectively. Randomly selected needle pairs are exchanged between the plans 401 and 403 to produce plans for the next generation, shown in FIGS. 4C and 4D as 401′ and 403′, with needle positions 405′ and 407′.

In mutation, needles can be mutated off or onto the plan. A needle can be mutated off the plan, i.e., it can be eliminated from the plan and transferred to the over-supply inventory. A location for a needle can be mutated on if the over-supply (OS) inventory (which contains those needles mutated off) has a needle suitable for implantation at that location. If mutated on, the needle is removed from the OS inventory.

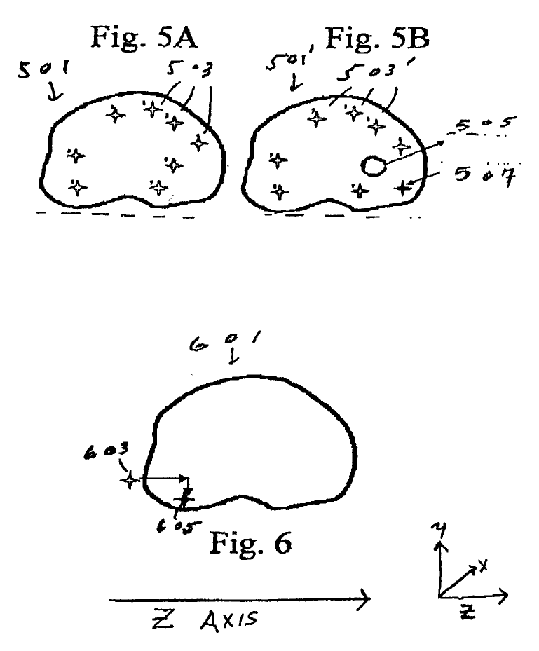

Mutation off and on will be explained with reference to FIGS. 5A and 5B. FIG. 5A shows a plan 501 having needle locations 503. FIG. 5B shows a mutated plan 501′ whose needle locations 503′ differ from the previous needle locations 503 in that one needle location 505 has mutated off the plan and in that another needle location 507 has mutated onto the plan. Mutation is different from merely relocating a needle in that the needles at locations 505 and 507 can have different characteristics, e.g., different numbers or types of seeds or different spacing between seeds.

Needle migration can take place in one or more of the x, y, and z directions in discrete increments. The increments are determined by the template that will be used, described later. Containment within the prostate is required; that is to say, the migration is constrained such that a needle may not migrate such that one or more of the seeds falls outside of the prostate. The number of increments migrated can be fixed (e.g., 1, 2, etc.) or randomized. In the latter case, randomization can be either uniform or Gaussian (or otherwise) distributed.

FIG. 6 shows a plan 601 in which a needle location has migrated in the x and y directions from a first location 603 to a second location 605. Migration in the z direction would change the needle offset, sometimes known as the retraction distance.

Containment can be represented by x, y, z1 and z2, or any indexing/labeling method to define the depth (e.g., from z1 to z2) of prostate tissue at each potential needle location (e.g., x, y). A planned needle is said to be contained if the location of the needle in the x-,y-plane falls within the contours of the prostate and the depth of the prostate tissue at its given location is greater than or equal to the total length of the seed train (e.g., from the center of the 1^{st} seed to the center of the last seed).

Weak containment may be defined in the x/y and z directions if the needle passage is within a predefined distance from the prostate periphery, base and apex, respectively (or more generally from a location within, or on the boundary of, the prostate or from the boundary of the prostate). Any form of weak containment may be preferred by the practitioner to the strict containment described in the previous paragraph, and the methods of the present invention are still applicable without further modification.

Containment is used in centering the preplan. To initially map a preplan onto the intraoperatively updated 3D shape and size of the prostate, the method of centering is applied. In that method, the preplan is shifted in the x, y and z directions as needed until the number of radioactive source trains that achieve containment is maximized.

Each needle has a predefined pattern of radioactive sources vs. spaces, or a predefined length of active material. The spacing between radioactive sources is not necessarily uniform or consistent. Radioactive sources contained within each train may be of different strength (e.g., being of higher activity on either end) or indeed of different isotopes (e.g., I-125 vs. Pd-103). To speed up computation, it is convenient (but not necessary) to pre-compute the dosage pattern of the needle for table look-up during repeated calculation. Tabulation of the dosage in that case may be conveniently done in the cylindrical coordinate system.

In addition to radioactive sources, or as an alternative thereto, a multitude of other therapeutic agents may be effective in the medical practice of interstitial implantation. Those include (but are not limited to) heating and cooling energy delivery by such means as radio frequency, microwave, magnetic resonance, freezing apparatus, as well as drugs, viral and gene vectors, and such like. Each method has a unique therapeutic agent delivery pattern, which may be pre-computed and tabulated for table look-up during repeated calculations -similar to the case of a needle containing radioactive sources of a given type and pattern. Thus the same principles may be applied in each circumstance.

The total needle inventory includes the preplanned set of needles (the “Preplan Inventory,” or “PP” inventory) with their associated patterns for the spacing of the sources within each needle plus the OS inventory. The PP inventory is that determined by the practitioner to be appropriate for the prostate size/shape and disease to be treated, and may be planned using one of several optimization or inverse planning methods (including but not limited to PIPER-I), or indeed by manual or nomogram look-up methods. The OS inventory includes an additional number of preloaded needles, which the practitioner wishes to have on hand at the time of the implantation to afford greater flexibility. The number of needles in the OS inventory can be specified by the practitioner (or recommended by the computer planning system) and can be zero or any positive integer; the methods of the present invention ensure that optimal dosimetry will be generated using primarily the PP inventory and secondarily the OS inventory. The needles in the OS inventory may be specified by the practitioner as containing any number and type of radioactive sources with any spacing of the sources within each needle. Indeed, when the practitioner is prepared to load the needles in the OR or use a manual radioactive source implantation applicator (e.g., Mick Applicator) the number of additional needles and their loading pattern (the spacing of radioactive sources within the needle) need not be specified. In the most general case, the OS inventory is represented in computer software as needles, each with a single radioactive source, which can be multiply loaded at each template location.

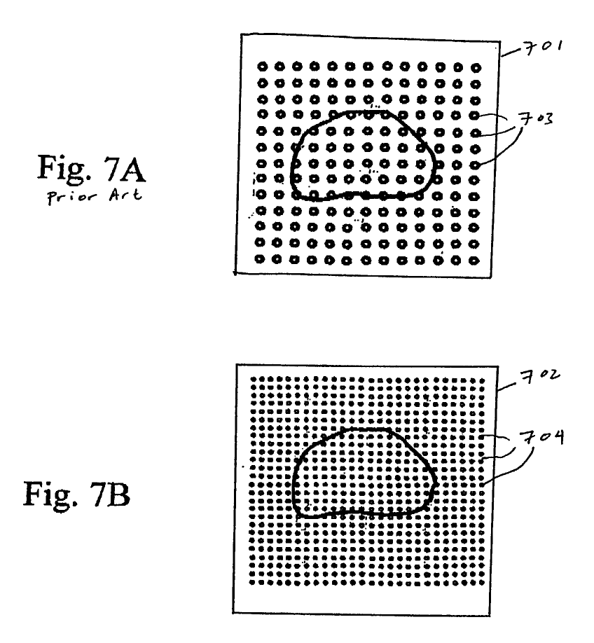

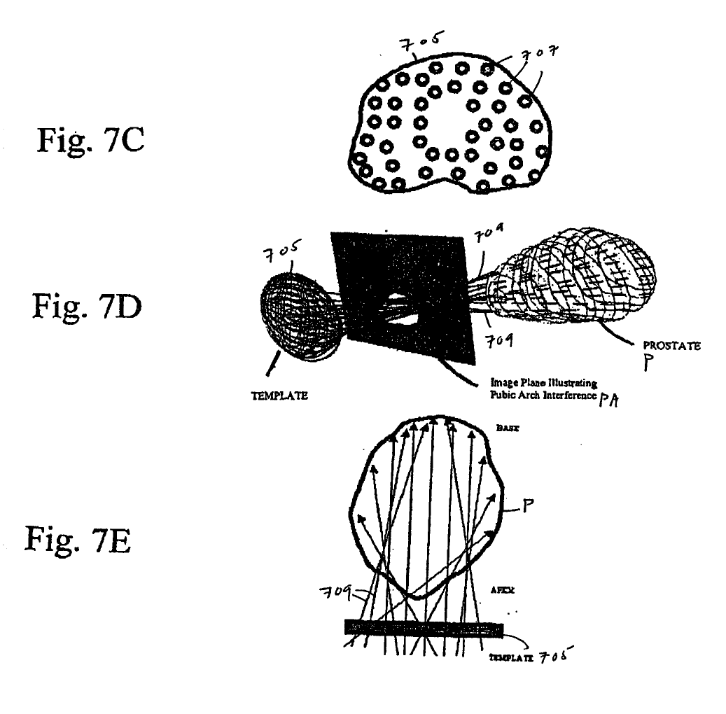

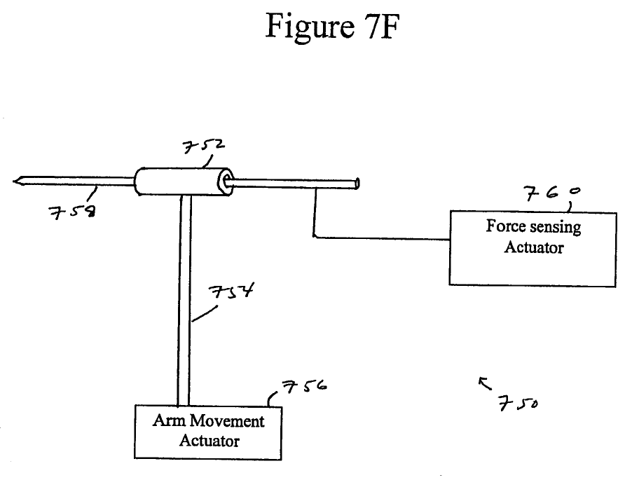

Another way of increasing the degree of optimization achievable is through the use of enhanced template technology, which will be explained with reference to FIGS. 7A-7E. As shown in FIG. 7A, the rectilinear templates 701 currently in use in prostate brachytherapy procedures are essentially flat plates with a 13×13 array of needle insertion holes 703 perpendicular to the plane of the template and spaced 0.5 cm on center. Since needles can only be placed where there is a hole in the template, the ability of PIPER-I (or any other planning system) to produce an optimized plan for needle and seed placement is constrained by the number, position and perpendicular angle of the holes in the template.

To overcome the above-noted deficiencies, a preferred embodiment of the present invention uses enhanced template (ET) technology which improves the degree of optimization possible In a simple embodiment of ET technology, the degree of potential optimization is improved by decreasing the distance between potential needle placement positions and increasing their number, as shown in FIG. 7B. For instance, a 26×26-hole template 702 can be designed with 2.5 mm between each needle insertion hole 704. That 26×26-hole template would cover the same surface area as the conventional 13×13-hole template but would have four times as many potential needle insertion positions. In the most general form, as shown in FIGS. 7C-7E, the ET 705 can be designed with a multiplicity of non-uniformly spaced static holes 707 which allow needles 709 to be inserted into the prostate P at various angles (to overcome such obstructions as the pubic arch PA) and with no constraints with regard to needle insertion position, as shown in FIGS. 7C-7E. Such templates afford the greatest degree of freedom for optimization under constraint.

Another preferred embodiment uses the virtual template. The Virtual Template (“VT”) is an extension of the conventional template and the ET described above in which the needle insertion hole(s) is not static, but is free to move. As shown in FIG. 7F, a preferred embodiment of the VT 750 has a single needle passage (e.g., needle hole) 752 mounted on a rigid stabilizing arm 754, which can be moved either manually or automatically (motor driven) in at least one angular degree of freedom or more specifically in 6 degrees of freedom: translations in the x, y, and z directions and rotations along the sagittal, coronal and anterior-posterior axes. In a preferred embodiment of the present invention, the VT is driven into the correct position and orientation by motor articulation by an arm movement actuator 756 based on the spatial information in the intraop plan for each needle insertion. Thereupon the corresponding needle 758 is inserted through the VT either by manual operation of the clinician, or by a force-sensing actuator 760, to the intended depth of the treated organ. The force-sensing actuator 760 applies motion in the forward direction as well as rotation along the needle axis (e.g., drilling motion) at a range of speeds and rotation patterns (clockwise, counterclockwise, or alternating). The force-sensing actuator 760 is interlocked for safety reasons by its range of travel and penetrating force, both of which can be set by the operator.

The VT 750 provides the greatest freedom for placing needles containing therapeutic material of any type and combination into the target organ while avoiding intervening obstructions and critical or normal anatomical structures. That preferred embodiment of the present invention allows optimized planning of needle placement in an unrestricted space afforded by the VT 750.

The present invention can be used in the absence of a preplan, such as being directly used in the OR, or indeed used to generate the preplan itself. To apply the methods of the invention, it is only necessary to predefine the number and pattern of needles/seeds or such other therapeutic agents, or their mixture (i.e., the total Needle Inventory). That aggregate of needles is then applied to the target organ (e.g., prostate) without the guide of the preplan, using either completely random distributions or one or more rules of thumb (e.g., peripheral loading, etc.). Mutation, migration and crossover (or any other computer optimization method) then progressively optimize the dosimetry to the desired endpoint.

As a special case, the practitioner may wish to use a generic Needle Inventory (not specified by generation of a preplan) for a particular range of prostate sizes. The methods of the present invention can then be applied to generate dosimetric plans that are customized to the shape and size of the prostate to be treated and use needles from the Needle Inventory. A set of such generic Needle Inventories may be used to cover the entire range of prostate sizes.

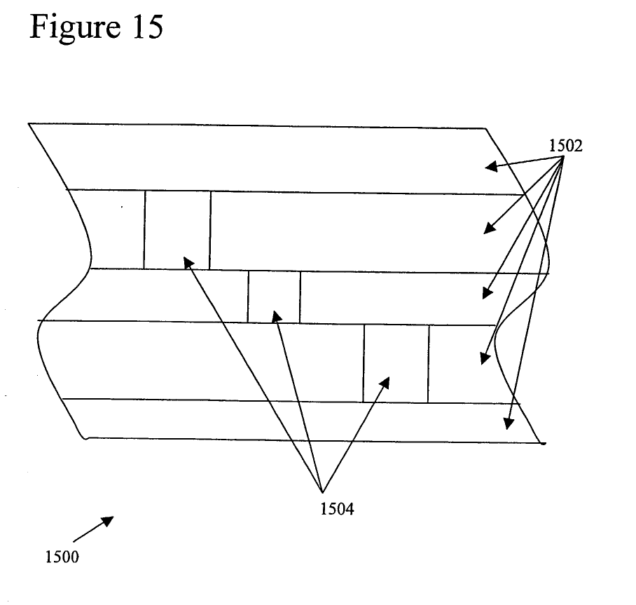

The present invention can also be used with pre-constructed seed carriers. Certain polymers, e.g., polylactic acid (PLA), polyglycolic acid (PGA) and their copolymers (PGLA), can be made into a rigid casing, which is bio-compatible and is bio-degraded once implanted into tissue in a consistent time period. It is possible to deliver seeds and/or other therapeutic agents or their mixture encased in such a carrier into the target organ (e.g., prostate) under the technologies of the present invention. The degradation schedule ranges from 0.5-16 mos. depending on material composition. During that time period, the seeds (etc.) maintain their planned relative configuration while delivering their therapeutic effect. As shown in FIG. 15, timed release of therapeutic, radio-sensitizing or tumor-modifying agents is possible by designing the carrier casing (shown in a partial cross-sectional view as 1500) using layers 1502 of different material compositions. As the layers 1502 bio-degrade, different therapeutic agents 1504, which can be any therapeutic agents described above or any other suitable therapeutic agents, are released at different times. The materials and thicknesses of the layers 1502 are selected to effect the appropriate timed releases of drugs, radio-sensitizing agents, or the like. Of course, different kinds of therapeutic agents 1504 can be used in the same carrier 1500.

To apply the methods of the present invention, it is convenient to pre-compute and tabulate the dose release schedule and range for table look-up in repeated calculations. Seeds delivered in suture material (such as Rapid Strand) can be treated as a simple example of that more general form of pre-constructed seed carrier, and the present methods may be applied accordingly.

Although a genetic algorithm is outlined in detail to generate optimized needle placement plans from a predefined set of seeds/needle patterns (i.e. a preloaded needle set), the present invention does not necessarily rely on using the genetic algorithm for optimization. Having defined the Needle Inventory consisting of the PP Inventory and the OS Inventory, or simply the total Needle Inventory, a number of well-known computer optimization methods can be applied. Some of those methods are briefly outlined as follows. Obviously, any method to arrange the Needle Inventory in the three-dimensional space of the target organ in a satisfactory way can be used.

Simulated Annealing (SA): That is a well-known optimization method in common use in brachytherapy. An initial temperature is assigned to a relatively high value, which translates into higher random operations (e.g., due to Gaussian distribution). Such random operations may include (but are not limited to) migration of needles and addition/deletion of needles, as described earlier. The temperature is assumed to decrease according to a “cooling schedule.” In addition, the temperature may be increased periodically and then allowed to decrease according to such schedule. Thus the SA is an iterative method of optimization, similar to GA, designed to overcome finding locally optimal but globally sub-optimal solutions.

Ad Hoc (or Brute Force) Optimization: The PP Inventory is first applied to the updated 3D target organ shape. Each needle is migrated in turn in a trial-and-error fashion, and the resulting dosimetry is examined for any improvement. The migration is kept if dosimetry improvement occurs; otherwise, the migration is not allowed to take place. Similarly, each needle is examined for possible removal into the OS Inventory for improved dosimetry, and likewise each potential needle location is examined for possible addition of a needle from the OS Inventory.

Hybrid Optimization: Any combination of genetic algorithm, simulated annealing, downhill simplex (or any other well-known optimization algorithm) or ad hoc methods, either in sequence or concurrently, may be used under the present invention to achieve the desired effect.

Specification of the total Needle Inventory, or the PP Inventory and the OS Inventory, is usually through a computer software user interface, where the practitioner defines all the variable parameters for each needle, such as seed locations, isotope type and strength, other therapeutic agent type and concentration, timed release schedules, etc. However, if a preplan is performed using the PIPER software system or using an embodiment of the present invention, all such information can be transmitted from the preplan to the intraoperative setting, or indeed to the manufacturer(s) of the needles, seeds, etc. An example of such information transmission may be through magnetic disk, diskette, cartridge, or optical storage media, as well as such devices as bar code scanning, network transfer or wireless transfer of data. Through the coding system, use of the pre-load optimization feature can be limited to radioactive sources (seeds) or pre-loaded needle sets provided by a specific manufacturer. Even if PIPER is not used for preplanning, the preloaded needle information can be imported to an embodiment of the present invention using one of those methods of transmission or by manually keying the data directly into the system. An alternative method of transmission is through a secured database server, which is maintained by the manufacturer of the preloaded needles or indeed of loose seeds, where the practitioner will be able to upload and download such information in conjunction with the specific purchase of therapeutic materials.

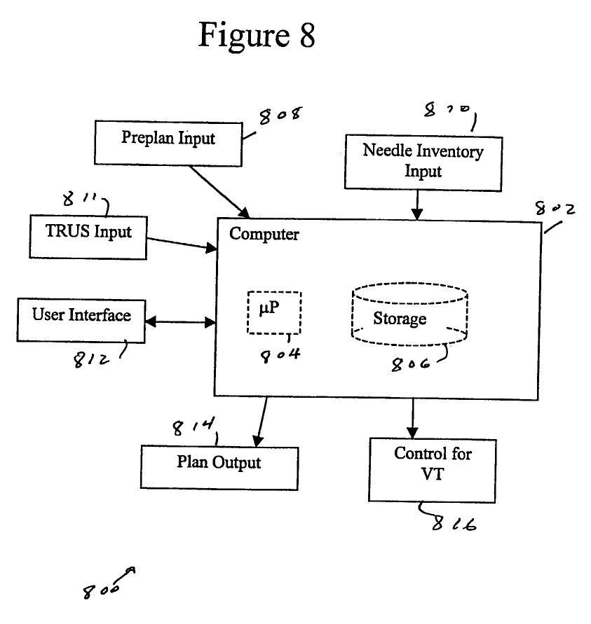

Any of the preferred embodiments, or other embodiments, can be implemented on the system of FIG. 8. The system 800 is based on a computer 802 having a microprocessor 804 (Pentium II or higher, PowerPC, or the like) and a hard drive and/or other persistent storage 806. It will be understood that the various inputs and outputs shown in FIG. 8 can be implemented on the same or different physical or logical components as needed.

As described above, the computer 802 receives an input of the preplan through a preplan input 808, an input of the needle inventory through a needle inventory input 810 and an input of the current position, size and shape of the prostate through a TRUS or other suitable input 811. The computer performs the processing described above, during which time it is under the control of the clinician through a user interface 812 (one or more of a monitor, keyboard, mouse, etc.). The computer can output the intraop plan through a plan output 814 and/or control the Virtual Template (VT) of FIG. 7F, if the VT is used, through a VT control output 816. In addition, or alternatively, an output can be provided to control any other suitable device, such as a video tube.

The system 800 does not have to use a preplan input 808. Instead, the system 800 can operate without a preplan. In such a case, the system 800 can randomly generate an initial plan which is used in place of the preplan, or it can generate the preplan as well as the intraop plan. The randomly generated initial plan can be based on a pre-loaded needle set. Multiple such needle sets can be available for various prostate sizes.

The needle inventory input 810 can also be used in accordance with a coding system which transmits information regarding the therapeutic agents. If the system 800 operates without a preplan, the system can use the information to generate either the random plan or the preplan. The information can also be used to limit use of the system to sources supplied by a particular manufacturer. Some practitioners may still prefer to pre-load their own needles. Those users enter a code which is supplied with each batch of loose seeds from the designated manufacturer in order to utilize the pre-load optimization feature of the system 800. For those practitioners who purchase pre-loaded needles from the designated manufacturer, the code is used to transmit information about the pre-loaded needle set to the system 800.

Experimental results will now be set forth.

Data from fourteen patients who had participated in an earlier study of the PIPER brachytherapy planning system were used for this test. Six (6) of the patients had been implanted with I-125 seeds and eight (8) with Pd-103 seeds. The prostate anatomy of each of these patients had been imaged by transrectal ultrasound several weeks prior to the brachytherapy procedure. The prostate anatomy of each patient had been imaged again in the operating room during the brachytherapy procedure. Thus, two sets of anatomic data were available for each patient: (1) the prostate anatomy as imaged and contoured during the preplanning visit and (2) the prostate anatomy as imaged and contoured at the time of the brachytherapy procedure. The availability of this data allowed us to test whether re-optimization of a preloaded set of needles produced a plan that was demonstrably superior to implementation of the preplan without modification.

Three cases were constructed for each patient:

1. Preplan: This is the historical plan that was generated by the UR-1 PIPER prototype system during the initial study. The inverse planning engine of the PIPER prototype system was used to generate an optimized preplan for each patient, based on the prostate anatomy as contoured during the preplanning visit.

2. Non Optimized Preplan: To simulate implementation, the Preplan was centered on the prostate anatomy as contoured in the operating room and applied without changes. This is essentially what clinicians do when they align the template over the prostate during a “preplanned” brachytherapy procedure.

3. Optimized Preloaded Plan: PIPER's pre-load module was used to generate a plan using needle loadings and the prescribed minimum peripheral dose (mPD) from the Preplan, but optimized to the anatomy observed in the OR. (In addition to the preplan needles, five additional needles “preloaded” with 10 seeds were included in an “overstock” inventory to facilitate optimization. This is consistent with the practice of most brachytherapists who order approximately 10% more seeds than a plan requires to accommodate changes. Other input of the overstock inventory has also been tested but the data that follow do not show those results.)

Data generated for each plan included: the minimum peripheral dose (mPD), dose-volume histogram (DVH) curves and the isodose distribution.

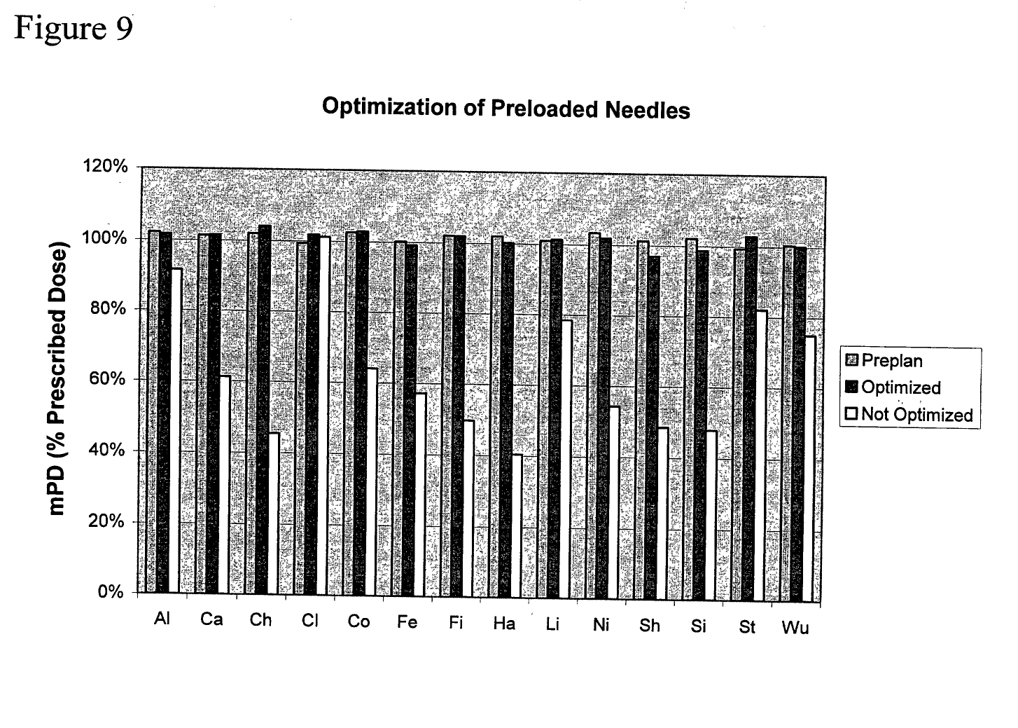

FIG. 9 shows the minimum peripheral dose data for all 14 patients. The data is normalized to percentage of the prescribed minimum peripheral (mPD) dose so that patients receiving I-125 seeds (typical prescribed mPD=145 Gy for monotherapy, 120 Gy for boost) and Pd-103 seeds (typical mPD=115 Gy for monotherapy, 90 Gy for boost), whether monotherapy or boost, can be displayed on the same chart.

It is immediately apparent from the data in the FIG. 9 that direct implementation of the preplan without optimization achieves only a fraction of the prescribed minimum peripheral dose. In 7 of the 14 cases examined, the minimum peripheral dose of the un-optimized preplan was less than 60% of the prescribed dose. In contrast, the optimized preloaded plan achieved a minimum peripheral dose that was, in each case, very close to 100% of the prescribed dose, and almost identical to that of the Preplan itself. The data clearly demonstrate that PIPER's genetic algorithm based planning engine can be utilized to optimize the placement of a set of preloaded needles and that the result obtained is superior to utilization of preplan needles without optimization.

In fact, this result is so striking that one must ask the following. If the standard technique of implementing a preplan without change is so bad, why does the standard preplan technique work at all? And, how does the standard preplan technique manage to achieve procedure outcomes that have been reported to be as good as those achieved by radical prostatectomy?

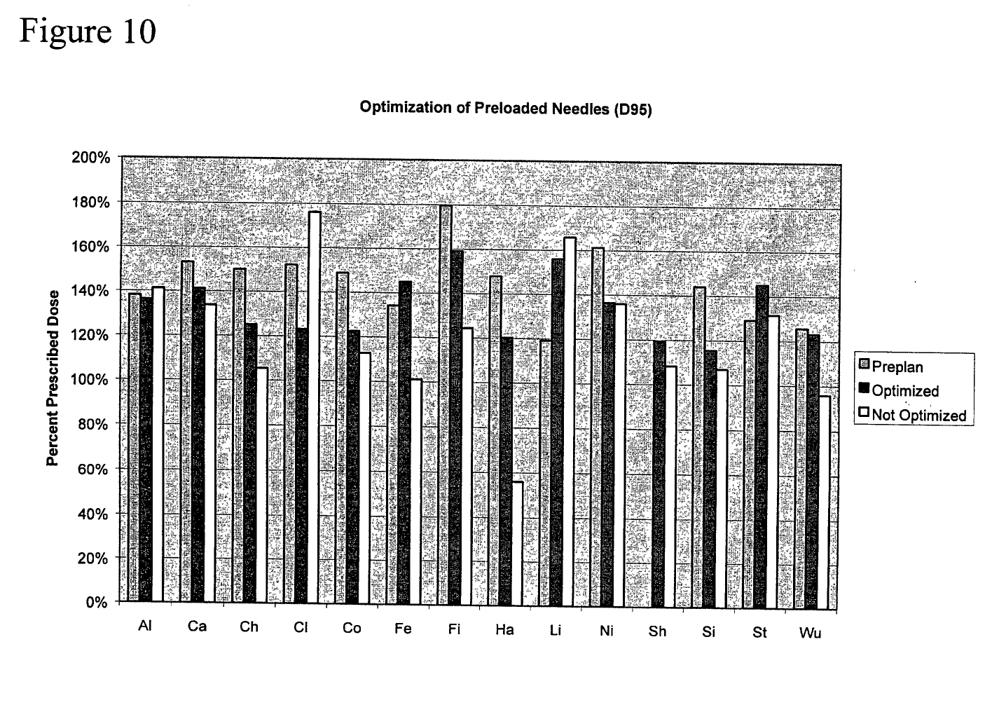

As shown in FIG. 10, the answer to these questions can be ascertained by examination of the D95 data (i.e. the percentage of the minimum prescribed dose delivered to 95% of the volume of the prostate). Without optimization, in 13 of the 14 patient data sets, 95% of the prostate volume receives a dose of radiation equivalent to or greater than 96% of the minimum prescribed dose. In the 14^{th} patient, the D95 dose is only 56% of the prescribed minimum peripheral dose. In other words, in most cases, with the standard preplan technique, only a small fraction of the total prostate volume does not receive the prescribed dose. To the extent that this fraction is small and does not contain malignant cells, the standard preplan technique can be expected to be efficacious. However, to the extent that malignant cells are located in this small portion of the prostate, and do not receive a killing dose of radiation, brachytherapy can be expected to fail. By contrast, with optimization as in the preplan itself, 100% of the prostate volume receives the prescribed dose (FIG. 9), and D95 values are consistently greater than 120% of the prescribed dose (FIG. 10). (Note that the D95 presented in FIG. 10 is not entirely the same as the D95 reported in the brachytherapy literature by practitioners based on postimplant CT dosimetry. FIG. 10 assumes that all seeds are precisely placed in their intended locations. In reality, seed displacements commonly observed tend to further lower the D95, thus causing a greater percentage of the preplanned cases to be under-dosed at the D95 level.)

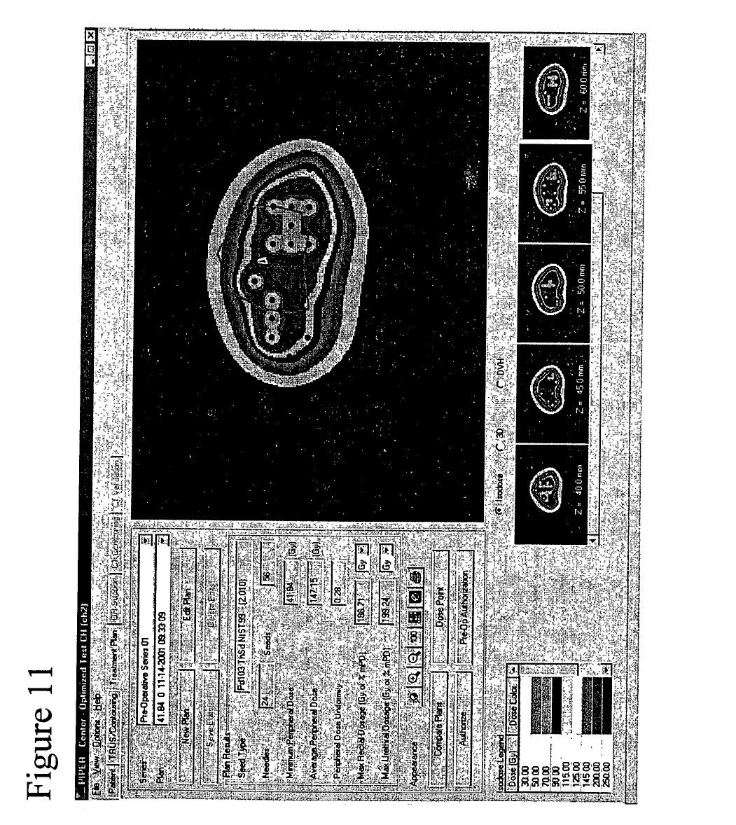

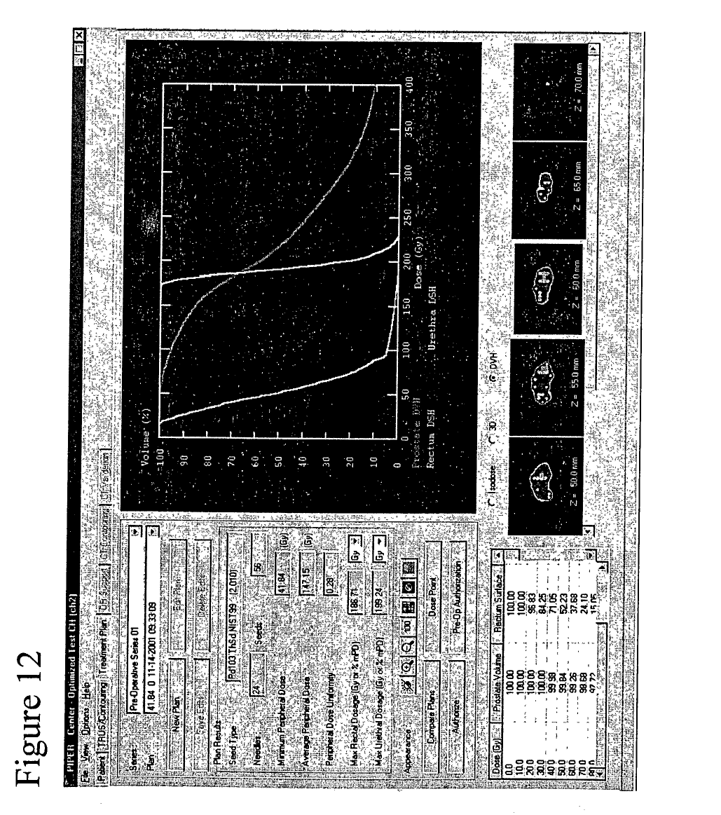

FIGS. 11 and 12 present isodose and DVH data respectively for a plan that has not been subsequently re-optimized in the OR at the time of implantation with PIPER's Pre-load technology, for patient CH and the isotope Pd-103. The isodose data (FIG. 11) illustrate that without optimization a portion of the prostate does not receive the prescribed minimum peripheral dose of 90 Gy. Without optimization, the actual minimal peripheral dose is 41.84 Gy. Also note that without optimization several seeds are implanted outside the prostate capsule. Seeds implanted outside the prostate capsule in the vicinity of sensitive structures e.g. the neurovascular bundles and rectum can cause serious complications.

The corresponding DVH data for this same patient (CH—Not Optimized) are presented in FIG. 12. Note that the minimum peripheral dose delivered to the prostate is less than the prescribed dose of 90 Gy.

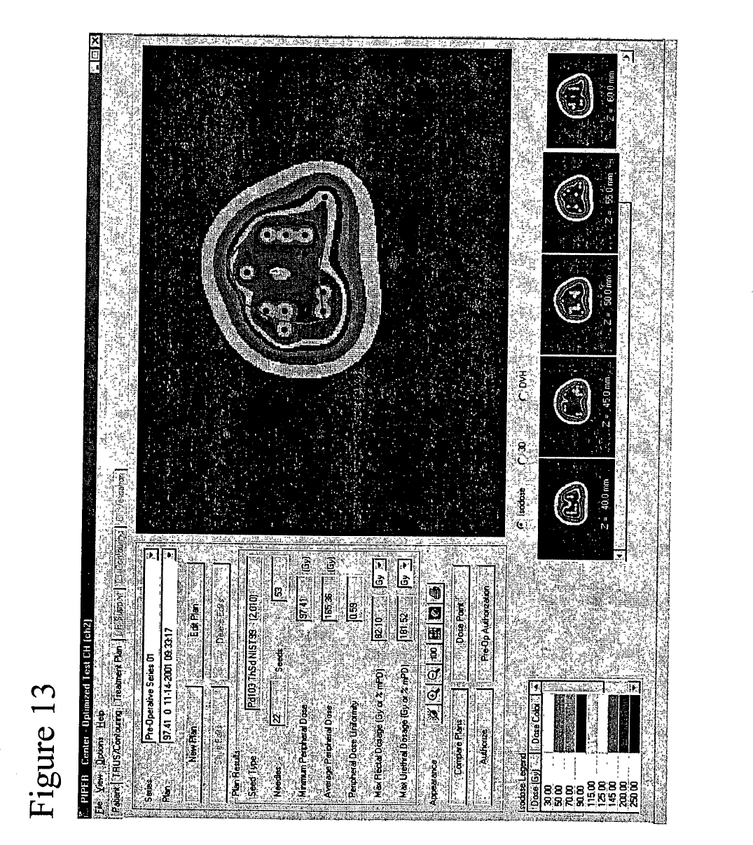

FIG. 13 presents isodose data for the same prostate section from the same patient (CH) as in FIG. 11. The results presented in FIG. 13 show that with optimization (using the PIPER pre-load technology) the entire prostate receives 100% of the prescribed dose of 90 Gy. One seed is implanted outside the prostate. (FIGS. 11 and 13 both show the prostate boundary as a line in the large isodose plot.) (In this test version of the software, the implantation of seeds outside the prostate capsule was not disallowed. Logic preventing extra-capsular implantation can be easily added to the optimization algorithm.)

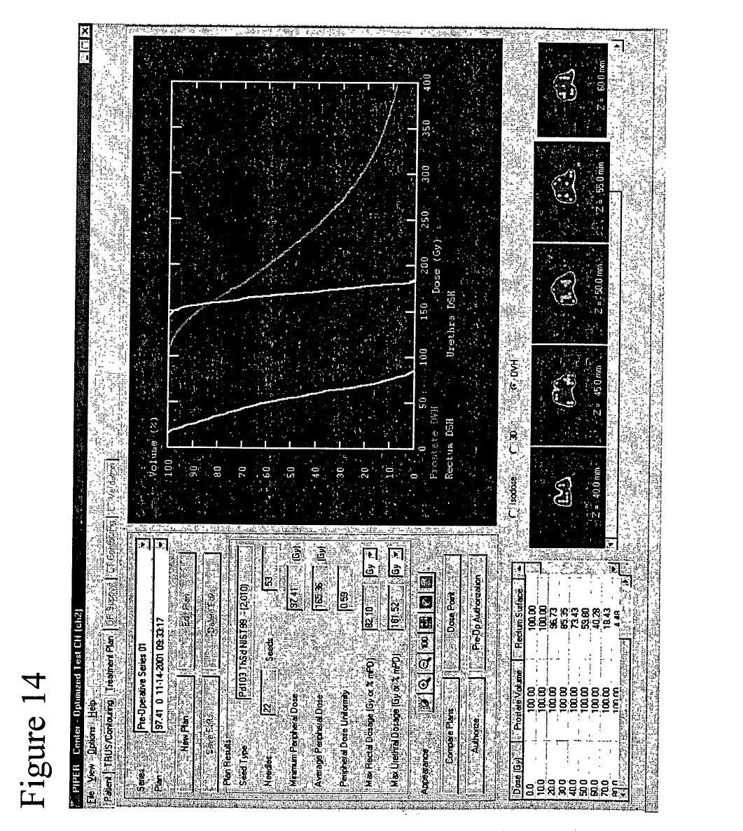

The corresponding DVH data for this same patient (CH—Optimized) are presented in FIG. 14. In comparison to the DVH data presented in FIG. 12 (same patient, but not optimized), note that optimization of the plan at the time of implantation improves the dose profiles of the prostate, urethra and rectum. The minimum dose delivered to the prostate is increased to prescription levels; while the maximum dose delivered to the urethra and rectum are reduced below the results obtained without optimization.

Plans vs. Outcomes: Needle deviations, seed migration and other implementation problems can be expected to degrade the quality of all plans when they are actually applied in the OR. Because of their superior dose coverage, optimized plans can be expected to be more tolerant of implementation problems than plans that have not been optimized.

In summary, the Pre-Load Optimization technology was tested in the UR-1 prototype PIPER System. Using data from 14 patients the Pre-Load Optimization technology was shown to be capable of generating optimized plans for the placement of preloaded needles. The minimum peripheral dose of each of the optimized plans created was nearly equivalent to the prescribed minimum peripheral dose (average mPD=101%±2%). The isodose distribution of individual slices in each plan was in agreement with the mPD data.

When preplans were applied without optimization the resulting minimum peripheral dose delivered to the prostate was only 64% (±19%) of the prescribed dose. Inspection of the isodose distribution of individual slices in the non-optimized plans confirmed that portions of the prostate received considerably less than the minimum peripheral dose.

These data show that the Pre-Load Optimization technology will be able to provide practitioners with the plan optimization benefits associated with intraoperative planning and the efficiency and convenience associated with preloaded needles.

While various preferred embodiments of the present invention have been set forth above in detail, those skilled in the art who have reviewed the present disclosure will readily appreciate that other embodiments can be realized within the scope of the present invention. Therefore, the present invention should be construed as limited only by the appended claims.

We claim:

1. A method of planning for a therapy in which a plurality of therapeutic agents are inserted into an organ to be treated, the method comprising:
(a) forming an initial plan for insertion of the plurality of therapeutic agents into the organ;
(b) when the plurality of therapeutic agents are to be inserted, determining at least one of a position, a size and a shape of the organ; and
(c) modifying the initial plan to conform to said at least one of the position, the size and the shape of the organ.

2. The method of claim 1, wherein the initial plan is a preplan, and wherein step (a) is performed before step (b).

3. The method of claim 2, wherein the organ is a prostate.

4. The method of claim 2, wherein a coding system is used to transmit information regarding the therapeutic agents to a planning system which performs steps (b) and (c), and wherein steps (b) and (c) are performed in accordance with the information transmitted by the coding system.

5. The method of claim 4, wherein the coding system is used to limit the plurality of therapeutic agents to therapeutic agents provided by a specific vendor.

6. The method of claim 3, wherein the plurality of therapeutic agents comprise radioactive sources.

7. The method of claim 6, wherein:
the radioactive sources are pre-loaded into needles;
step (a) comprises determining a plurality of insertion locations for the needles; and
step (c) comprises at least one of: (i) moving at least one of the insertion locations, (ii) not utilizing at least one of the insertion locations or (iii) adding at least one additional insertion location.

8. The method of claim 7, wherein a coding system is used to transmit information regarding at least one of a radioactive source type, a source manufacturer and specifications of the pre-loaded needle set to a planning system which performs steps (b) and (c), and wherein steps (b) and (c) are performed in accordance with the information transmitted by the coding system.

9. The method of claim 8, wherein the coding system is used to limit the therapeutic agents to radioactive sources and/or pre-loaded needle sets provided by a specific vendor.

10. The method of claim 7, wherein step (c) is performed using a genetic algorithm.

11. The method of claim 10, wherein the genetic algorithm comprises a crossover.

12. The method of claim 10, wherein the genetic algorithm comprises a mutation.

13. The method of claim 12, wherein the mutation comprises a deletion of one of the needles.

14. The method of claim 12, wherein the mutation comprises an addition of a further one of the needles.

15. The method of claim 10, wherein the genetic algorithm comprises a migration of at least one of the insertion locations.

16. The method of claim 15, wherein the migration is constrained such that all of the radioactive seeds are contained within the organ.

17. The method of claim 15, wherein the migration is constrained such that said at least one of the insertion locations is within a predetermined distance of a location within or on a boundary of the organ or within a predetermined distance of the boundary of the organ.

18. The method of claim 7, wherein step (c) is performed using simulated annealing.

19. The method of claim 7, wherein step (c) is performed using numerical optimization.

20. The method of claim 6, wherein dosage patterns of the radioactive sources are pre-computed and stored for use in step (c).

21. The method of claim 20, wherein the dosage patterns are stored in a lookup table.

22. The method of claim 1, wherein step (c) comprises modifying the preplan into a plan in which the plurality of therapeutic agents are inserted into the organ through holes in a template.

23. The method of claim 22, wherein the holes in the template are spaced less than 5 mm on center.

24. The method of claim 22, wherein the holes in the template are spaced in a non-rectilinear arrangement.

25. The method of claim 22, wherein the template comprises a moveable needle passage.

26. The method of claim 25, wherein the template further comprises a movable stabilizing arm supporting the moveable needle passage.

27. The method of claim 1, wherein step (c) is performed on a computer.

28. The method of claim 27, wherein the computer stores an inventory of the therapeutic agents, and wherein step (c) is performed in accordance with the inventory.

29. The method of claim 28, wherein the inventory includes an oversupply inventory of the therapeutic agents for addition to the initial plan.

30. The method of claim 28, wherein the inventory comprises a plurality of inventories for different sizes of the organ.

31. The method of claim 28, wherein the inventory comprises an inventory specific to the patient whose organ is to be treated.

32. The method of claim 27, wherein the computer also performs step (a).

33. The method of claim 32, wherein the computer generates the initial plan randomly.

34. The method of claim 33, wherein the computer stores an inventory of the therapeutic agents and generates the initial plan randomly in accordance with the inventory.

35. The method of claim 1, wherein the therapeutic agents are encased in at least one carrier which is bio-degraded when the at least one carrier is inserted into the organ.

36. The method of claim 35, wherein the therapeutic agents comprise radioactive seeds.

37. The method of claim 36, wherein the therapeutic agents further comprise therapeutic agents other than radioactive seeds.

38. The method of claim 35, wherein the at least one carrier is constructed to provide timed release of at least one of the therapeutic agents.

39. The method of claim 1, wherein step (c) is performed using a genetic algorithm.

40. The method of claim 1, wherein step (c) is performed using simulated annealing.

41. The method of claim 1, wherein step (c) is performed using numerical optimization.

42. The method of claim 1, wherein the plurality of therapeutic agents comprise a viral vector.

43. The method of claim 1, wherein dosage patterns of the plurality of therapeutic agents are pre-computed and stored for use in step (c).

44. The method of claim 43, wherein the dosage patterns are stored in a lookup table.

45. A system for planning for a therapy in which a plurality of therapeutic agents are inserted into an organ to be treated, the system comprising:
an input for receiving information about at least one of a position, a size and a shape of the organ; and
a processor, in communication with the input, for receiving an initial plan for insertion of the plurality of therapeutic agents into the organ and for modifying the initial plan to conform to said at least one of the position, the size and the shape of the organ.

46. The system of claim 45, wherein:
the therapeutic agents comprise radioactive sources pre-loaded into needles;
the initial plan comprises a plurality of insertion locations for the needles; and
the processor modifies the initial plan by moving at least one of the insertion locations, not utilizing at least one of the insertion locations or adding at least one additional insertion location.

47. The system of claim 46, wherein the processor modifies the initial plan by using a genetic algorithm.

48. The system of claim 47, wherein the genetic algorithm comprises a crossover.

49. The system of claim 47, wherein the genetic algorithm comprises a mutation.

50. The system of claim 49, wherein the mutation comprises a deletion of one of the needles.

51. The system of claim 49, wherein the mutation comprises an addition of a further one of the needles.

52. The system of claim 47, wherein the genetic algorithm comprises a migration of at least one of the insertion locations.

53. The system of claim 52, wherein the migration is constrained such that all of the radioactive seeds are contained within the organ.

54. The system of claim 52, wherein the migration is constrained such that said at least one of the insertion locations is within a predetermined distance of a location within the organ.

55. The system of claim 46, wherein the processor modifies the initial plan by using simulated annealing.

56. The system of claim 46, wherein the processor modifies the initial plan by using numerical optimization.

57. The system of claim 46, further comprising storage, in communication with the processor, in which dosage patterns of the radioactive sources are precomputed and stored.

58. The system of claim 49, wherein the dosage patterns are stored in a lookup table in the storage.

59. The system of claim 45, further comprising a template having holes for insertion of the therapeutic agents into the organ.

60. The system of claim 59, wherein the holes in the template are spaced less than 5 mm on center.

61. The system of claim 59, wherein the holes in the template are spaced in a non-rectilinear arrangement.

62. The method of claim 59, wherein the template comprises a movable needle passage.

63. The system of claim 62, wherein the template further comprises a movable stabilizing arm supporting the movable a needle passage.

64. The system of claim 45, further comprising storage, in communication with the processor, for storing an inventory of the therapeutic agents, and wherein the processor modifies the initial plan in accordance with the inventory.

65. The system of claim 64, wherein the inventory includes an oversupply inventory of the therapeutic agents for addition to the initial plan.

66. The system of claim 64, wherein the inventory comprises a plurality of inventories for different sizes of the organ.

67. The system of claim 64, wherein the inventory comprises an inventory specific to the patient whose organ is to be treated.

68. The system of claim 45, further comprising an input for receiving the initial plan.

69. The system of claim 45, wherein the processor also generates the initial plan.

70. The system of claim 69, wherein the processor generates the initial plan randomly.

71. The system of claim 70, further comprising storage for storing an inventory of the therapeutic agents, and wherein the processor generates the initial plan randomly in accordance with the inventory.

72. The system of claim 45, wherein the processor modifies the initial plan by using a genetic algorithm.

73. The system of claim 45, wherein the processor modifies the initial plan by using simulated annealing.

74. The system of claim 45, wherein the processor modifies the initial plan by using numerical optimization.

75. The system of claim 45, further comprising storage, in communication with the processor, in which dosage patterns of the plurality of therapeutic agents are pre-computed and stored.

76. The system of claim 76, wherein the dosage patterns are stored in a lookup table in the storage.

77. A virtual template for use in insertion of needles carrying therapeutic agents into an organ to be treated, the virtual template comprising:
a needle passage member having a needle passage for insertion of one of the needles; and
a stabilizing arm for holding the needle passage member in a position and for being moved to vary the position for each of the needles.

78. The virtual template of claim 77, in which the stabilizing arm is movable in at least one angular degree of freedom.

79. The virtual template of claim 78, wherein the stabilizing arm is movable in three linear degrees of freedom and three angular degrees of freedom.

80. The virtual template of claim 78, further comprising an arm movement actuator for moving the stabilizing arm.

81. The virtual template of claim 77, further comprising a force-sensing actuator for inserting the needles.

82. The virtual template of claim 77, further comprising an actuator for inserting each of the needles while rotating said each of the needles about a needle axis.

83. The virtual template of claim 82, wherein the actuator rotates said each of the needles in a single rotational direction.

84. The virtual template of claim 82, wherein the actuator rotates said each of the needles in alternation between clockwise and counterclockwise directions.

85. A method for inserting needles carrying therapeutic agents into an organ to be treated, the method comprising:
(a) developing a plan specifying locations at which the needles are to be inserted into the organ; and
(b) inserting the needles into the organ at the locations using a template which does not have a rectilinear arrangement of holes.

86. The method of claim 85, wherein the template has a non-rectilinear arrangement of said holes.

87. The method of claim 85, wherein:
the template comprises a needle passage member having a needle passage for insertion of one of the needles and a stabilizing arm for holding the needle passage member in a position and for being moved to vary the position for each of the needles in accordance with the locations specified in the plan; and
step (b) comprises moving the stabilizing arm for insertion of each of the needles.

88. A therapeutic agent carrier for insertion into an organ to be treated, the carrier comprising:
a coating which bio-degrades when inserted into the organ; and
a therapeutic agent for treating the organ, the therapeutic agent being exposed when the coating bio-degrades.

89. The carrier of claim 88, comprising a plurality of the therapeutic agents and a plurality of the coatings for being bio-degraded in succession to expose successive ones of the therapeutic agents.

90. The carrier of claim 89, wherein the plurality of therapeutic agents comprise a radioactive seed.

91. The carrier of claim 89, wherein the plurality of therapeutic agents comprise a radio-sensitizing agent.

92. The carrier of claim 89, wherein the plurality of therapeutic agents comprise a drug.
